(19) United States
(12) Patent Application Publication
(10) Pub. No.: US 2022/0310243 A1
(43) Pub. Date: Sep. 29, 2022

(54) CUSTOMIZATION OF AN ORTHOPAEDIC IMPLANT

(71) Applicant: The Johns Hopkins University, Baltimore, MD (US)

(72) Inventors: Jarred A. BRESSNER, Baltimore, MD (US); Mikhail OSANOV, Baltimore, MD (US); James K. GUEST, Lutherville Timonium, MD (US)

(73) Assignee: The Johns Hopkins University, Baltimore, MD (US)

(21) Appl. No.: 17/597,221

(22) PCT Filed: Jul. 22, 2020
(86) PCT No.: PCT/US2020/043079

§ 371 Date: Dec. 29, 2021

Related U.S. Application Data
(60) Provisional application No. 62/881,245, filed on Jul. 31, 2019.

(51) Int. Cl. G16H 40/63; G16H 10/60; G16H 30/20

(52) U.S. Cl. CPC G16H 40/63; G16H 10/60; G16H 30/20

(57) ABSTRACT

An implant customization platform may receive image data associated with a bone of a patient. The implant customization platform may convert the image data to a structural representation of the bone. The implant customization platform may identify, based on the structural representation, a placement for an orthopaedic implant relative to the bone. The implant customization platform may determine a performance characteristic for a combination of the bone and the orthopaedic implant. The implant customization platform may determine, using an implant customization model, a data representation of the orthopaedic implant based on the structural representation, the placement, and the performance characteristic. The implant customization platform may perform an action associated with the data representation to permit the orthopaedic implant to be formed.

### CROSS-REFERENCE TO RELATED APPLICATION

This patent application claims priority to U.S. Provisional Patent Application No. 62/881,245, filed on Jul. 31, 2019, and entitled “CUSTOMIZATION OF AN ORTHOPAEDIC IMPLANT.” The disclosure of the prior application is considered part of and is incorporated by reference into this patent application.

### GOVERNMENT LICENSE RIGHTS

This invention was made with U.S. Government support under grant T32 training grant (T32 AR067708), awarded by the National Institute of Health (NIH)/Department of Health and Human Services (DHHS). The U.S. Government has certain rights in the invention.

### BACKGROUND

Orthopaedic implants provide support and/or rehabilitation to a bone and/or skeletal structure of a patient. For example, a bone implant may be fused with a bone to ensure that the bone heals properly, grows properly, and/or the like. An arthroplasty implant may be a bone implant that is fused with a bone that is associated with a joint of a patient (e.g., a shoulder, a knee, a hip, and/or the like), to rehabilitate and/or replace a portion of the joint. An interbody orthopaedic implant, such as an interbody spine implant, may be configured to provide support between one or more bones (e.g., vertebra) of a patient.

### SUMMARY

According to some implementations, a method may include receiving profile information associated with a patient, wherein the profile information indicates that the patient is to receive an orthopaedic implant associated with a bone of the patient; receiving image data associated with the bone, wherein the image data is associated with a computed tomography scan of the bone; converting the image data to a structural representation of the bone, wherein the structural representation corresponds to a bone structure of the bone; analyzing the image data to identify structural characteristics of the bone structure; identifying, based on the structural characteristics, a placement for the orthopaedic implant relative to the bone; determining a performance characteristic for a combination of the bone and the orthopaedic implant; determining, using an implant customization model, a data representation of the orthopaedic implant based on the structural representation, the placement, and the performance characteristic; and performing an action associated with the data representation to permit the orthopaedic implant to be formed.

According to some implementations, a device may include one or more memories and one or more processors, communicatively coupled to the one or more memories, to: receive profile information associated with a patient; receive a three-dimensional rendering of a bone of the patient; generate, from the three-dimensional rendering, a structural representation of the bone; determine, based on the structural representation, a placement for an orthopaedic implant relative to the bone; determine, from the profile information, a performance characteristic for the orthopaedic implant; determine, using an implant customization model, a data representation of the orthopaedic implant based on the structural representation and the placement, wherein the implant customization model is configured to optimize the performance characteristic; and perform an action associated with the data representation to permit the orthopaedic implant to be formed.

According to some implementations, a non-transitory computer-readable medium may store one or more instructions. The one or more instructions, when executed by one or more processors of a device, may cause the one or more processors to: receive image data associated with a bone of a patient; convert the image data to a structural representation of the bone; identify, based on the structural representation, a placement for an orthopaedic implant relative to the bone; determine a performance characteristic for a combination of the bone and the orthopaedic implant; determine, using an implant customization model, a data representation of the orthopaedic implant based on the structural representation, the placement, and the performance characteristic; and perform an action associated with the data representation to permit the orthopaedic implant to be formed.

### BRIEF DESCRIPTION OF THE DRAWINGS

FIGS. 1A-1C are diagrams of one or more example implementations described herein.

FIG. 2 is a diagram of an example environment in which systems and/or methods described herein may be implemented.

FIG. 3 is a diagram of example components of one or more devices of FIG. 2.

FIGS. 4-6 are flowcharts of one or more example processes for customizing an orthopaedic implant as described herein.

### DETAILED DESCRIPTION

The following detailed description of example implementations refers to the accompanying drawings. The same reference numbers in different drawings may identify the same or similar elements.

The biomechanical function of an orthopaedic implant is often limited due to a large discrepancy between material and structural properties of the orthopaedic implant and the native bone of the patient. An orthopaedic implant is generally manufactured from a relatively complex and/or expensive manufacturing process (e.g., a molding process, a diecasting process, and/or the like) due to the orthopaedic implant being comprised of relatively strong materials such as stainless steel, a titanium alloy, a cobalt-chromium-molybdenum alloy, and/or the like. Accordingly, for orthopaedic implants that are to integrate with bone (referred to herein, generally, as a “bone implant” and/or, more specifically, as an “arthroplasty implant” when the orthopaedic implant is used in association with a joint of the patient) the difference in stiffness between a primarily or fully metallic orthopaedic implant and bone may cause stress shielding (especially in cases of arthroplasty implants) where the orthopaedic implant and bone are fused together. Stress shielding decreases a patient's bone density (and/or prevents bone growth) near the bone implant due to the patient's bone naturally relying on the bone implant for strength and/or to withstand force. Such a decrease in bone density may lead to further injury to the patient and/or re-injury of the bone and/or a joint associated with the bone due to the weakening of the bone at the point of fusion with the bone implant.

In many instances, an orthopaedic implant is used in interbody spine fusions, whereby disk space between vertebral bodies (or “vertebrae”) is eliminated and the vertebral bodies, themselves, may be promoted to be fused via the orthopaedic implant (referred to herein as an “interbody fusion implant”). In such cases, the orthopaedic implant is to provide stability to the spine of the patient while facilitating fusion of the vertebral bodies. Issues with spine fusions arise from the development of adjacent segment degeneration and/or proximal junction kyphosis. Such issues are caused by the fusion of two vertebrae together, forming a fused spinal segment that is a relatively larger mass in the spine that creates a greater moment of inertia and/or moment arm in the spine and promotes stress concentrations to build up on adjacent segments. Such stress concentrations may lead to progressive and/or persistent pain for the patient, neural compromise, and/or further surgery to stabilize other segments of the spine.

Moreover, the above issues with orthopaedic implants are exacerbated by the fact that individual patients have unique bone properties (e.g., no two patients are the same). In previous techniques, due to the above-mentioned complexity of manufacturing orthopaedic implants, when selecting an orthopaedic implant for a patient, an off-the-shelf implant is selected (e.g., by size) regardless of potentially significant incompatibilities with respect to geometry, material, and/or structural properties of the bone or bones of the patient (e.g., because the complexity requires mass production of off-the-shelf implants).

According to some implementations described herein, an implant customization platform may determine a configuration for (e.g., may design) a custom orthopaedic implant based on patient-specific bone structure and/or profile information. Furthermore, as described herein, the implant customization platform may permit an orthopaedic implant to be designed (e.g., a topology optimization model, a machine learning model, and/or the like) and/or formed (e.g., assembled, manufactured, and/or the like) using materials that are capable of providing optimal (according to the data model) stiffness, flexion, strength, fatigue strength, tailored stress distribution, permeability, and/or the like (e.g., composite materials, and/or the like). In some implementations, as described herein, the implant customization platform may, from a structural representation of a patient's bone, determine a placement for an orthopaedic implant relative to the bone, determine, based on a performance characteristic associated with the patient and using the data model, a data representation of the orthopaedic implant, and use the data representation to enable the orthopaedic implant to be formed or manufactured and/or to enable the orthopaedic implant to be surgically received by the patient.

In this way, the implant customization platform enables a custom orthopaedic implant to be designed and/or formed for a particular patient (and/or a group of patients with one or more of the same characteristics described herein) and to achieve specific performance characteristics in combination with a bone structure of the patient. For example, a bone implant, for a particular patient, that is designed and/or formed in association with the examples described herein, may be configured to reduce (relative to previous techniques) or prevent stress shielding associated with the orthopaedic implant after implantation in the patient. Additionally, or alternatively, an interbody fusion implant, for a particular patient, designed and/or formed in association with the examples described herein, may be configured to more closely approximate the native stresses of a healthy spine. Accordingly, the implant customization platform, as described herein, may be configured to design an orthopaedic implant that can be formed and that, after being received by a patient, reduces the risks of further injury, risks of re-injury, and/or risks of needing an additional or subsequent surgery.

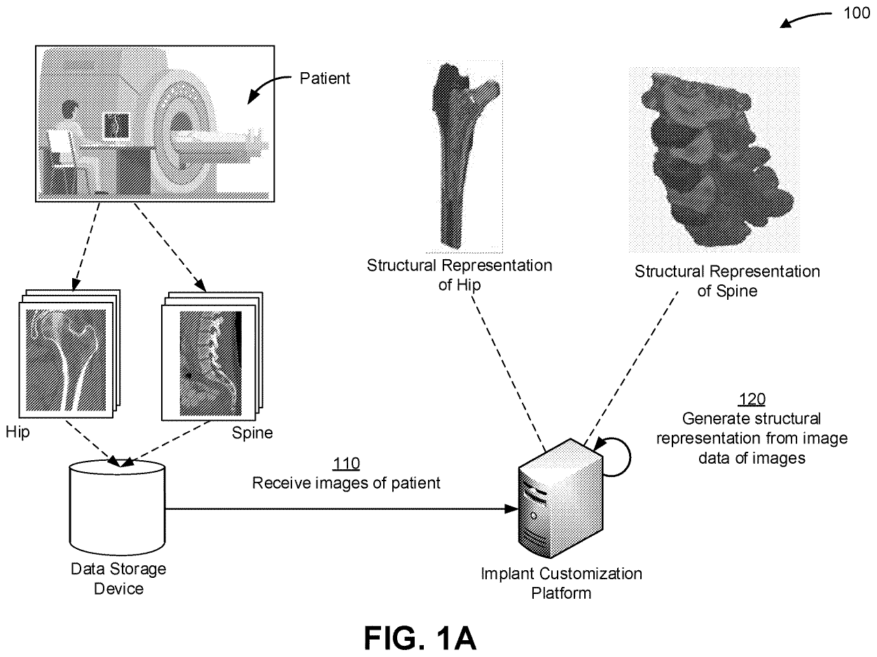

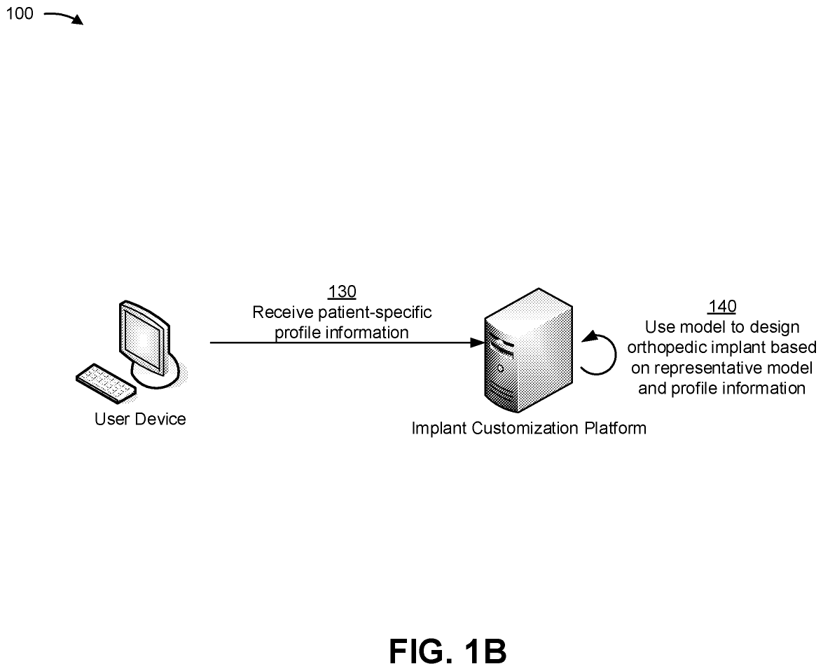

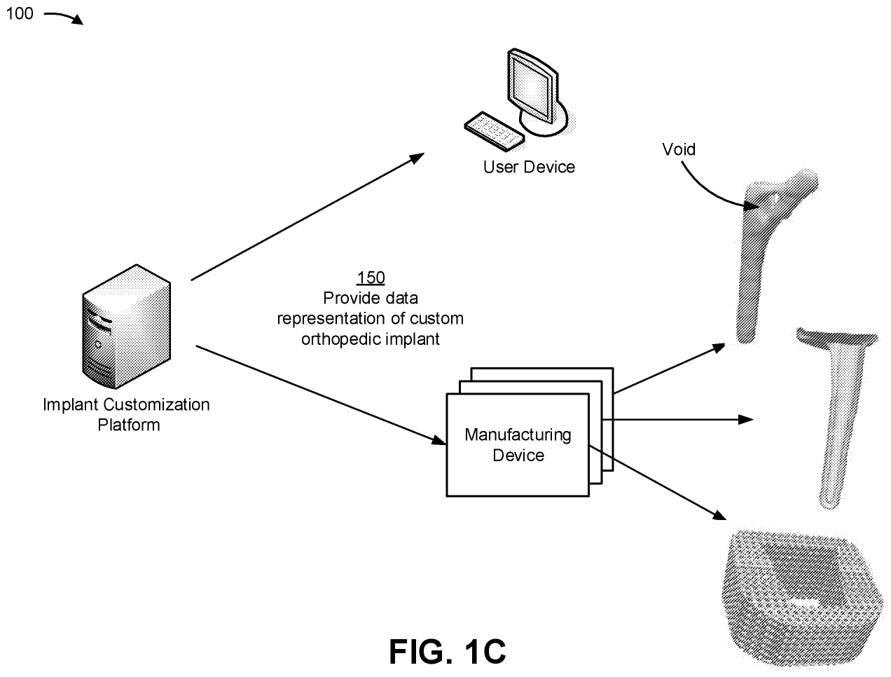

FIGS. 1A-1C are diagrams of an example implementation 100 described herein. Example implementation 100 includes an implant customization platform, a medical imaging device, a data storage device, a user device, and one or more manufacturing devices (referred to individually as a “manufacturing device” and collectively as “manufacturing devices”) that may be used in accordance with designing and/or forming a custom (e.g., patient-specific) orthopaedic implant (which may be referred to as an “orthopaedic implant” in the following examples), as described herein. According to some implementations, the implant customization platform may receive image data associated with a bone of the patient, determine a structural representation of the bone, determine a placement for the orthopaedic implant relative to the bone, and determine a data representation corresponding to a configuration of the orthopaedic implant according to the structural representation, the placement, and one or more performance characteristics associated with the patient. Additionally, or alternatively, the implant customization platform may provide the data representation to a user device to enable a user to view and/or analyze the configuration of the orthopaedic implant (e.g., before and/or during surgery to implant the orthopaedic implant) and/or to permit a manufacturing device to form the orthopaedic implant so that the orthopaedic implant can be received by the patient.

As shown in FIG. 1A, and by reference number 110, the implant customization platform receives images of a patient. The images of the patient may include images of a bone of the patient that is to be associated with the orthopaedic implant. As described herein, a particular bone may be associated with an orthopaedic implant in that the bone is to interact with the orthopaedic implant when the implant is implanted within the patient. For example, the bone may interact with the orthopaedic implant by fusing to the orthopaedic implant and/or by being supported by the orthopaedic implant. The images may be captured and/or provided (e.g., to the data storage device) by the medical imaging device and/or received from a data storage device. The data storage device may be associated with (e.g., communicatively coupled with, installed within, and/or the like) the medical imaging device, the user device, and/or the implant customization platform. The images and/or image data may be associated with a CT scan (e.g., obtained from a CT scan device), a magnetic resonance imaging (MRI) scan, and/or the like.

According to some implementations, the implant customization platform may receive the images as image data (e.g., data that can be used to render the images). In some implementations, the image data may be representative of a plurality (or series) of images of the patient and/or of a specific bone of the patient. Additionally, or alternatively, the image data may correspond to a three-dimensional (3D) graphical representation of the bone associated with the orthopaedic implant.

In this way, the implant customization platform may receive image data associated with images of a patient and/or a bone of a patient to permit the implant customization platform to generate a structural representation of the bone of the patient using the image data.

As further shown in FIG. 1A, and by reference number 120, the implant customization platform generates a structural representation from the image data of the images. For example, the implant customization platform may convert the image data to a structural representation that corresponds to a bone structure (e.g., a topology, a shape, a size, a bone density, and/or the like) of the bone. The structural representation may correspond to a representation of structural and/or morphological properties of the bone structure. As described herein, the image data may correspond to a 3D rendering (e.g., a 3D graphical representation) of the patient and/or the bone of the patient. Such a 3D rendering may be comprised of a plurality of voxels having corresponding graphical values (e.g., values of a color code (e.g., red-green-blue (RGB) values)) for particular portions of the patient.

According to some implementations, the implant customization platform may be configured to identify and/or extract image data for voxels that specifically depict bones of the patient (e.g., using an object recognition technique, an image processing model trained to identify and/or extract image data for bone of a patient, and/or the like). For example, the implant customization platform may use a computer vision technique, such as a convolutional neural network technique, to assist in classifying image data (e.g., image data including representations of bone of a patient, tissue of a patient, and/or the like) into a particular class. More specifically, the implant customization platform may determine that bone has a particular characteristic (e.g., is a particular shape, is a particular size, is a particular topology, is in a particular color range, and/or the like). On the other hand, the implant customization platform may determine that bones do not have a particular characteristic and/or that tissue of the patient does not have a particular characteristic. In some implementations, the computer vision technique may include using an image recognition technique (e.g., an Inception framework, a ResNet framework, a Visual Geometry Group (VGG) framework, and/or the like), an object detection technique (e.g. a Single Shot Detector (SSD) framework, a You Only Look Once (YOLO) framework, a cascade classification technique (e.g., a Haar cascade technique, a boosted cascade, a local binary pattern technique, and/or the like), and/or the like), an edge detection technique, an object in motion technique (e.g., an optical flow framework and/or the like), and/or the like.

Certain properties of the medical imaging device may cause the 3D rendering of the bone to be depicted with various shading and/or colors (e.g., due to a radio density captured in the images) that correspond to different bone densities in certain parts of the bone. For example, relatively darker shades of gray may represent lesser bone density than relatively lighter shades of gray. Accordingly, from the 3D rendering of the bone, the implant customization platform may convert graphical values of the voxels of the 3D rendering (e.g., graphical values corresponding to a radiodensity captured in the voxels of the 3D rendering) to property values (e.g., associated with a material density, associated with pores (e.g., porosity), associated with an elastic modulus, and/or the like) for voxels of the structural representation of the bone (e.g., a representation associated with particular loads, boundary conditions, and/or the like). The structural representation may be a model that can be analyzed to identify one or more structural (and/or morphological) characteristics (e.g., physical characteristics, such as topology, shape, size, density, stiffness, and/or the like) of the bone structure and/or portions of the bone structure. Additionally, or alternatively, the structural representation can be analyzed and/or manipulated, as described herein, to permit the implant customization platform to determine an optimal configuration for the orthopaedic implant.

In this way, the implant customization platform may generate a structural representation of the bone of the patient to permit the implant customization platform to determine a placement for the orthopaedic implant and/or an optimal configuration of the orthopaedic implant, as described herein.

As shown in FIG. 1B, and by reference number 130, the implant customization platform may receive patient-specific profile information from the user device. For example, the patient (or a representative of the patient) may provide information associated with the patient via the user device. Such information may include one or more profile characteristics of the patient (which may be referred to herein as “patient characteristics”), such as a past medical history, prescribed medications, a date of birth (or age), a height, a weight, a bone mineral density of one or more bones of the patient, a sex, a race, and/or the like.

In some implementations, the profile information may include information associated with one or more performance characteristics for the orthopaedic implant. For example, a user (e.g., the patient, a doctor or surgeon that is to implant the orthopaedic implant in the patient, and/or the like) may indicate one or more performance characteristics that the orthopaedic implant is to provide to the patient (after the orthopaedic implant is surgically implanted). The profile information may indicate that the combination of the bone and orthopaedic implant is to be able to withstand a threshold level of force, a threshold fatigue cycling (e.g., a useful lifespan, usage duration, and/or the like), and/or the like. Additionally, or alternatively, the profile information may indicate that the orthopaedic implant is to prevent a threshold level of stress shielding (e.g., to prevent a decrease in bone density, bone growth, and/or the like). In some implementations, the profile information may indicate that the orthopaedic implant is to have a particular porosity (e.g., which may be based on the type of implant, a level of fusion of the implant to the bone, an ability of the implant to receive bodily fluids or tissue, and/or the like).

According to some implementations, the profile information may indicate whether the orthopaedic implant is to be capable of administering a medical substance into the patient and/or into the bone of the patient. The orthopaedic implant may be configured to administer a medical substance as described in U.S. patent application Ser. No. ______, titled “ORTHOPAEDIC IMPLANT TO ADMINISTER A MEDICAL SUBSTANCE” and filed on MONTH, DATE, YEAR, which is hereby incorporated by reference. For example, the orthopaedic implant may be configured to have a dosing mechanism that is capable of releasing the medical substance through and/or from the orthopaedic implant (e.g., via porous openings in a structure of the orthopaedic implant). Such a medical substance may be a fluid and/or a solid (e.g., a powder) that is to provide treatment to the patient (e.g., to the bone of the patient, to interstitial areas of the patient's body, to tissue of the patient, to an organ of the patient, to a blood stream of the patient, and/or the like). For example, the medical substance may be one or more medicines, one or more antibiotics, one or more supplements, one or more stimulants, and/or the like. Because such an ability may affect the structural integrity of the orthopaedic implant, a doctor may indicate whether or not the orthopaedic implant is to have such a capability, and if so, one or more particular structures for, mechanisms for, and/or types of medical substance releasing capabilities (which may have respective effects on the structural integrity of the orthopaedic implant) that may be used to provide such a capability.

Additionally, or alternatively, the profile information may include information associated with an activity level associated with a desired performance capability of the patient. Such an activity level and/or performance capability may be based on a scoring system and/or grade that can be based on activities (e.g., that have corresponding degrees of exertion, performance requirements, and/or the like) that are to be performed (and/or are preferred to be performed) by the patient after recovering from a surgery to implant the orthopaedic implant in the patient. As a specific example, a patient's desire to be able to play a competitive sport may correspond to a relatively high activity level while a patient's desire to (at a minimum) be able to walk may correspond to a relatively low activity level. Such an activity level may be representative of and/or correspond to an expected load on the orthopaedic implant (e.g., once implanted) and/or the combination of the bone and the orthopaedic implant. Additionally, or alternatively, the activity level may correspond to an expected load on a skeletal structure of the patient, which may be affected by the orthopaedic implant, regardless of which bone of the skeletal structure is associated with the orthopaedic implant.

In this way, the implant customization platform may receive patient specific profile information that may indicate and/or identify one or more patient characteristics and/or preferences with respect to performance characteristics of the orthopaedic implant (and/or a combination of the orthopaedic implant and a bone of the patient).

As further shown in FIG. 1B, and by reference number 140, the implant customization platform uses the structural representation to design an orthopaedic implant based on the structural representation and the profile information of the patient. For example, the implant customization platform may determine, from the structural representation of the bone, a placement (or an approximate placement) for the orthopaedic implant in association with the bone (and/or whether some of the bone is to be removed and/or replaced by the orthopaedic implant). The placement may be based on one or more structural characteristics of the bone (e.g., which may be determined from the structural representation). For example, a certain orthopaedic implant may be configured to be fused with a bone and/or placed between bones at a particular location based on certain structural characteristics of the bone(s). Furthermore, as described herein, the implant customization platform may determine a data representation for an optimal configuration of the orthopaedic implant (e.g., according to a data model as described herein) based on one or more of the performance characteristics associated with the patient.

According to some implementations, the implant customization platform may be associated with and/or configured to use an implant customization model that includes or utilizes one or more data models to design an orthopaedic implant, as described herein. For example, the implant customization model may include an image processing model to analyze images of the bone of the patient, identify the bone of the patient, generate a graphical representation of the bone, determine structural characteristics of the bone based on the images and/or graphical representation, and/or the like. Additionally, or alternatively, the implant customization model may include a topology optimization model to design a physical configuration of the orthopaedic implant, including topology, shape, size, porosity, and/or the like. Such a topology optimization model may be configured specifically for a particular orthopaedic implant, based on one or more structural representations of the orthopaedic implant (e.g., similar to the generated structural representation of the bone of the patient), based on one or more parameters associated with the orthopaedic implant (e.g., porosity, stiffness, flexion, strength, fatigue strength, tailored stress distribution, permeability, and/or the like), and/or the like. Accordingly, using possible structural characteristics of the orthopaedic implant used to train and/or configure the topology optimization model, the implant customization model may determine an optimal configuration for the orthopaedic implant in association with the structural representation of the bone of the patient.

In some implementations, the implant customization model (e.g., via the topology optimization model) may be configured to iteratively modify (e.g., increase or decrease) property values (e.g., constitutive values corresponding to a structure represented by the voxel, a property of the structure represented by the voxel, a lack of structure represented by the voxel, a morphological characteristic represented by the voxel, and/or the like) for voxels of the structural representation of the bone and/or the structural representation of the orthopaedic implant to simulate various combinations of the bone and/or the orthopaedic implant. For each iteration, the implant customization model may analyze the simulated structural representations of combinations of the bone and the orthopaedic implant (at a determined placement for the orthopaedic implant) to correspondingly simulate an increase or decrease in stiffness, an increase or decrease in strength (e.g., an ability to withstand more or less force), an increase or decrease in porosity, an increase or decrease in potential stress shielding, an increase or decrease in a substance release capability, and/or the like. Accordingly, the implant customization model may alter one or more of the property values for the structural representation of the orthopaedic implant until the implant customization platform determines that the one or more performance characteristics are optimized (e.g., according to certain parameters of the topology optimization model, porosity, stiffness, flexion, strength, fatigue strength, tailored stress distribution, permeability, and/or the like). The implant customization platform may generate the data representation of the configuration of the orthopaedic implant from the property values for the structural representation that optimizes the one or more performance characteristics.

Additionally, or alternatively, the topology optimization model may be configured to forecast and/or predict changes to structural characteristics of the bone of the patient (e.g., based on length, which can be simulated relative to an amount of use of the orthopaedic implant and/or treatment provided to the bone), and determine corresponding effects on the remaining portions of the bone, the orthopaedic implant, and/or the skeletal structure of the patient. Accordingly, as described herein, the implant customization platform may utilize an implant customization model (e.g., including and/or associated with a topology optimization model) to determine alternative values for voxels of the structure and/or morphological representation (e.g., that can be replaced and/or altered by implanting the orthopaedic implant in association with the bone).

In some implementations, one or more artificial intelligence techniques, including machine learning, deep learning, neural networks, and/or the like can be used to identify a particular bone of a patient, determine the placement for the orthopaedic implant based on the structural representation, implement and/or update a data model (e.g., an image processing model, a topology optimization model, and/or the like) to design the orthopaedic implant, and/or the like. For example, the implant customization model of the implant customization platform may be a machine learning model to design an optimal configuration for an orthopaedic implant for a patient based on patient specific information and/or performance characteristics. In such cases, the implant customization platform may train the implant customization model based on one or more parameters associated with designing and/or configuring an orthopaedic implant, such as a type of the orthopaedic implant, profile information for a patient associated with the orthopaedic implant, a performance characteristic for the orthopaedic implant, and/or the like. The implant customization platform may train the implant customization model using historical data associated with designing and/or configuring other orthopaedic implants for other patients based on the above parameters and/or post-operative structural representations of a combination of the other orthopaedic implants and other patients (e.g., results of surgeries to implant the other designed orthopaedic implants). Using the historical data and the one or more parameters as inputs to the implant customization model, the implant customization platform may determine an optimal configuration (e.g., represented by a data representation of the orthopaedic implant) for an orthopaedic implant to permit the orthopaedic implant to be formed and implanted in the patient to improve the overall health of the patient, the bone of the patient, a joint associated with the bone of the patient, and/or an overall lifespan of the orthopaedic implant.

According to some implementations, the implant customization model of the implant customization platform may determine a structural representation based on a plurality of structural representations, generated as described herein. For example, a plurality of structural representations associated with a group of patients (e.g., patients having the same or similar patient characteristics and/or bone structures) may be generated as described herein. In such a case, the implant customization model may combine the plurality of structural representations to generate a single generic structural representation that is representative of the bones of the patients (and/or correspondingly, of a single orthopaedic implant for the bones of the patient). For example, the implant customization model of the implant customization platform may be a machine learning model to design an optimal configuration for an orthopaedic implant for a group of patients based on specific information and/or performance characteristics that are common to the individual patients of the group of patients. In this way, the implant customization model may design an orthopaedic implant that may be optimal for a plurality of patients and/or reusable across a plurality of patients (e.g., a group of patients with the same or similar profile information).

As described herein, the data representation may correspond to any data or structure of data (e.g., a file, such as an image file, a computer aided drawing (CAD) file, a computer aided manufacturing (CAM) file, an additive manufacturing file, and/or the like) that is representative of custom configuration of the orthopaedic implant. In some implementations, the data representation includes information identifying particular types of materials that are to be used for particular components and/or elements of the orthopaedic implant. For example, the particular types of materials may be indicated based on the determined property values for the structure representation of the orthopaedic implant in the optimized configuration of the orthopaedic implant. In other words, a particular property value for a voxel of the structure representation may represent and/or correspond (e.g., according to a manufacturing mapping) to a particular material or density of material or void that is to be utilized to form the corresponding portion of the orthopaedic implant.

In this way, the implant customization platform may determine, using an implant customization model, a data representation of the orthopaedic implant to permit the orthopaedic implant to be formed (e.g., by the manufacturing device) and/or implanted within the patient to enable the patient to engage in one or more activities in accordance with specific performance characteristics.

As shown in FIG. 1C, and by reference number 150, the implant customization platform provides the data representation of the orthopaedic implant to a user device and/or one or more of the manufacturing devices. Additionally, or alternatively, the implant customization platform may store the data representation in a data structure (e.g., to permit the data representation to be obtained by the user device and/or the manufacturing devices, to permit the data representation to be accessed and/or updated at a later time, and/or the like).

The implant customization platform may provide the data representation of the orthopaedic implant to the user device to permit the user device to present a graphical representation of the custom configuration of the orthopaedic implant via a user interface (e.g., a display) of the user device. Additionally, or alternatively, the data representation may be provided to the user device to permit a user (e.g., a surgeon, an orthopaedic implant specialist, and/or the like) to analyze and/or adjust one or more aspects of the orthopaedic implant. For example, the user device may include an application configured to interpret the data representation, display the custom configuration of the orthopaedic implant, and/or enable adjustments of the orthopaedic implant (e.g., adding a feature such as a particular substance release capability, adjusting flexibility, altering materials for use, and/or the like) and/or corresponding adjustments to the data representation.

In some implementations, the implant customization platform may cause the manufacturing device to form an orthopaedic implant, by providing the data representation to the manufacturing device and/or sending instructions (e.g., with the data representation) to cause the manufacturing device to form the orthopaedic implant. Each of the manufacturing devices may be configured for a particular type of implant (e.g., a hip implant, a knee implant, an interbody spine implant, and/or the like) or the same manufacturing device may be configured to create multiple types of implants.

In some implementations, a manufacturing device configured to form bone implants may be configured to form an orthopaedic implant from materials that enable a relatively high degree of stiffness, to mimic bone density (e.g., titanium alloys, cobalt chrome alloys, composite materials, and/or the like), have a particular a morphology (e.g., architected materials, lattices, foams), have a particular shape and/or feature, and/or the like. Such shapes and/or features may correspond to particular configurations of bones associated with the bone implants. Additionally, or alternatively, the manufacturing devices may be capable of forming bone implants that do not have configurations that mimic a bone of a patient. For example, the implant customization platform may determine that a particular hip implant for a patient is to have a void (as shown in FIG. 1C) to optimize a particular performance characteristic (e.g., based on the structural representation of the patient's bone, the structural representation of the hip implant with the void, and/or the performance characteristic). Accordingly, the manufacturing device may be configured to form a hip implant that includes a void despite the fact that a patient's hip bone may not include such a void.

Additionally, or alternatively, a manufacturing device configured to form interbody spine implants may be configured to form an orthopaedic implant to resemble trabecular bone (e.g., via a lattice structure, a unitary cell structure, a hollow structure, a trussed structure, a foam structure, a hydroxyapatetite coating, and/or the like). For example, such interbody spine implants may be composed of lattice elements that demonstrate locally varying volume fraction, and material allocated along a most ideal force flow (e.g., as determined by the implant customization platform). As described herein, the implant customization platform may design and/or the manufacturing device may be configured to form an interbody spine implant that includes a fully trabeculated implant, a peripherally solid structure that surrounds a trabecular portion, and/or the like. Such custom designed interbody spine implants may be improved over previous implants that have mechanical properties that are randomly generated as potential bulk behavior. In this way, the interbody spine implants, as designed by the implant customization platform and/or formed by the manufacturing devices, are targeted and structured so as to steer mechanical response of the entire vertebral segment (e.g., as determined by the implant customization model of the implant customization platform) to reduce stress concentrations and therefore reduce adjacent segment degeneration.

Accordingly, the manufacturing devices may be equipped with particular materials and/or capabilities to form and/or manufacture a particular orthopaedic implant according to the data representation determined and/or generated by the implant customization platform.

In this way, the implant customization platform may provide a data representation of the orthopaedic implant to permit details of the orthopaedic implant to be analyzed and/or displayed (e.g., via a user device, a surgical device, and/or the like) and/or to permit a manufacturing device to form and/or manufacture the orthopaedic implant. Therefore, the implant customization platform may enable the orthopaedic implant to be received (e.g., via the surgical device and/or a surgery performed by a surgeon) by the patient.

Accordingly, as described herein, the implant customization platform enables a custom orthopaedic implant to be designed (e.g., using an implant customization model) and/or formed (e.g., using an additive manufacturing device). The custom orthopaedic implant may be configured for a particular patient and to enable the patient to perform activities in accordance with specific performance characteristics expected from a combination of the orthopaedic implant and one or more bones of the patient. As described herein, the implant customization platform may enable design and formation of a patient-specific bone implant that reduces or prevents stress shielding associated with a bone of the patient after the bone implant is received by the patient. Further, the implant customization platform may enable design and/or formation of a patient-specific interbody fusion implant that is configured to replace and/or resemble trabecular bone between vertebrae of the spine and that is capable of reducing stresses in the spine after the patient receives the interbody fusion implant. Accordingly, the implant customization platform, as described herein, enables design and/or formation of an orthopaedic implant that, after being received by a patient, reduces risks of further injury to the patient, reduces risks of re-injury to a bone associated with the orthopaedic implant, and/or reduces risks of needing for an additional or subsequent surgery.

As indicated above, FIGS. 1A-1C are provided merely as one or more examples. Other examples may differ from what is described with regard to FIGS. 1A-1C.

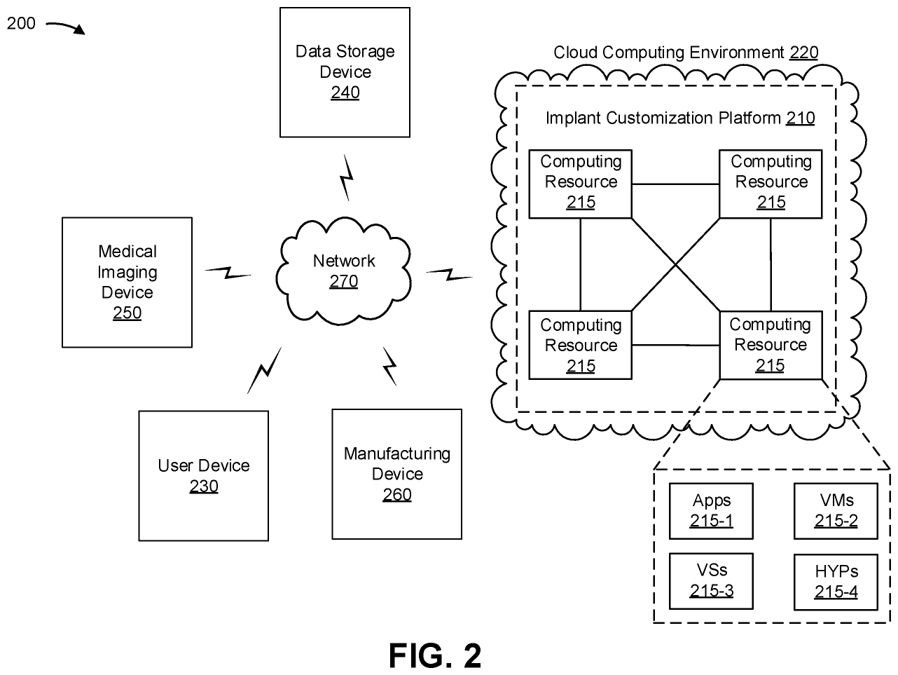

FIG. 2 is a diagram of an example environment 200 in which systems and/or methods described herein may be implemented. As shown in FIG. 2, environment 200 may include an implant customization platform 210 hosted by computing resources 215 of a cloud computing environment 220, a user device 230, a data storage device 240, a medical imaging device 250, a manufacturing device 260, and a network 270. Devices of environment 200 may interconnect via wired connections, wireless connections, or a combination of wired and wireless connections.

Implant customization platform 210 includes one or more computing resources 215 for designing a custom orthopaedic implant, as described herein. For example, implant customization platform 210 may be a platform implemented by cloud computing environment 220 that may analyze image data of a bone, determine a structural representation of the bone, and determine a configuration for a custom orthopaedic implant based on the structural representation of the bone and/or an orthopaedic implant to optimize a performance characteristic. In some implementations, implant customization platform 210 is implemented by computing resources 215 of cloud computing environment 220.

Implant customization platform 210 may include a server device or a group of server devices. In some implementations, implant customization platform 210 may be hosted in cloud computing environment 220. Notably, while implementations described herein may describe implant customization platform 210 as being hosted in cloud computing environment 220, in some implementations, implant customization platform 210 may be non-cloud-based or may be partially cloud-based.

Cloud computing environment 220 includes an environment that delivers computing as a service, whereby shared resources, services, and/or the like may be provided to user device 230 and/or manufacturing device 260. Cloud computing environment 220 may provide computation, software, data access, storage, and/or other services that do not require end-user knowledge of a physical location and configuration of a system and/or a device that delivers the services.

Computing resource 215 includes one or more personal computers, workstation computers, server devices, or another type of computation and/or communication device. In some implementations, computing resource 215 may host implant customization platform 210. The cloud resources described below may include compute instances executing in computing resource 215, storage devices provided in computing resource 215, data transfer devices provided by computing resource 215, and/or the like. In some implementations, computing resource 215 may communicate with other computing resources 215 via wired connections, wireless connections, or a combination of wired and wireless connections.

As further shown in FIG. 2, computing resource 215 may include a group of cloud resources, such as one or more applications (“APPs”) 215-1, one or more virtual machines (“VMs”) 215-2, virtualized storage (“VSs”) 215-3, one or more hypervisors (“HYPs”) 215-4, or the like.

Application 215-1 includes one or more software applications that may be provided to or accessed by user device 230 and/or manufacturing device 260. Application 215-1 may eliminate a need to install and execute the software applications on user device 230 and/or manufacturing device 260. For example, application 215-1 may include software associated with implant customization platform 210 and/or any other software capable of being provided via cloud computing environment 220. In some implementations, one application 215-1 may send/receive information to/from one or more other applications 215-1, via virtual machine 215-2.

Virtual machine 215-2 includes a software implementation of a machine (e.g., a computer) that executes programs like a physical machine. Virtual machine 215-2 may be either a system virtual machine or a process virtual machine, depending upon use and degree of correspondence to any real machine by virtual machine 215-2. A system virtual machine may provide a complete system platform that supports execution of a complete operating system. A process virtual machine may execute a single program and may support a single process. In some implementations, virtual machine 215-2 may execute on behalf of a user device (e.g., via user device 230), and may manage infrastructure of cloud computing environment 220, such as data management, synchronization, or long-duration data transfers.

Virtualized storage 215-3 includes one or more storage systems and/or one or more devices that use virtualization techniques within the storage systems or devices of computing resource 215. In some implementations, within the context of a storage system, types of virtualizations may include block virtualization and file virtualization. Block virtualization may refer to abstraction (or separation) of logical storage from physical storage so that the storage system may be accessed without regard to physical storage or heterogeneous structure. The separation may permit administrators of the storage system flexibility in how the administrators manage storage for end users. File virtualization may eliminate dependencies between data accessed at a file level and a location where files are physically stored. This may enable optimization of storage use, server consolidation, and/or performance of non-disruptive file migrations.

Hypervisor 215-4 provides hardware virtualization techniques that allow multiple operating systems (e.g., “guest operating systems”) to execute concurrently on a host computer, such as computing resource 215. Hypervisor 215-4 may present a virtual operating platform to the guest operating systems and may manage the execution of the guest operating systems. Multiple instances of a variety of operating systems may share virtualized hardware resources.

User device 230 includes one or more devices capable of receiving, generating, storing, processing, and/or providing information associated with an orthopaedic implant that is designed by implant customization platform 210, as described herein. For example, user device 230 may include a communication and/or computing device, such as a laptop computer, a tablet computer, a handheld computer, a desktop computer, a surgical device (e.g., for use in surgery to implant the orthopaedic implant), a mobile phone (e.g., a smart phone, a radiotelephone, and/or the like), a wearable communication device (e.g., a smart wristwatch, a pair of smart eyeglasses, and/or the like), or a similar type of device.

Data storage device 240 includes one or more devices capable of receiving, generating, storing, processing, and/or providing information associated with images (e.g., CT images) of a patient, image data associated with images of a patient, a structural representation associated with images of a patient and/or corresponding image data, and/or the like. For example, in some implementations, data storage device 240 may include a server device, a hard disk device, an optical disk device, a solid-state drive (SSD), a compact disc (CD), a network attached storage (NAS) device, a flash memory device, a cartridge, a magnetic tape, and/or another device that can store and provide access to perioperative images, demographic data, patient outcome metrics, and/or the like.

Medical imaging device 250 includes one or more devices capable of receiving, generating, storing, processing, and/or providing information and/or images (e.g., pre-operative images, intra-operative images, and/or post-operative images, and/or the like). For example, medical imaging device 250 may include a CT scan device, an MRI device, an X-ray device, a positron emission tomography (PET) device, an ultrasound imaging (USI) device, a photoacoustic imaging (PAI) device, an optical coherence tomography (OCT) device, an elastography imaging device, and/or a similar type of device. In some implementations, medical imaging device 250 may generate and provide one or more images and/or image to implant customization platform 210.

Manufacturing device 260 may include one or more devices capable of forming, assembling, and/or manufacturing an orthopaedic implant based on information (e.g., a data representation of the orthopaedic implant) generated by and/or received from implant customization platform 210. For example, manufacturing device 260 may include an additive manufacturing device (e.g., a 3D printer), a milling device, a selective laser melting (SLM) device, one or more assembly devices (e.g., one or more robotic machines, one or more mechanical devices, one or more molding or casting devices, and/or the like), and/or the like that are capable of receiving (e.g., via a communication device of manufacturing device 260) a data representation that is representative of a physical configuration of an orthopaedic implant and forming, assembling, and/or manufacturing the orthopaedic implant from the data representation.

Network 270 includes one or more wired and/or wireless networks. For example, network 270 may include a cellular network (e.g., a long-term evolution (LTE) network, a code division multiple access (CDMA) network, a 3G network, a 4G network, a 5G network, another type of next generation network, and/or the like), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, a cloud computing network, and/or the like, and/or a combination of these or other types of networks.

The number and arrangement of devices and networks shown in FIG. 2 are provided as one or more examples. In practice, there may be additional devices and/or networks, fewer devices and/or networks, different devices and/or networks, or differently arranged devices and/or networks than those shown in FIG. 2. Furthermore, two or more devices shown in FIG. 2 may be implemented within a single device, or a single device shown in FIG. 2 may be implemented as multiple, distributed devices. Additionally, or alternatively, a set of devices (e.g., one or more devices) of environment 200 may perform one or more functions described as being performed by another set of devices of environment 200.

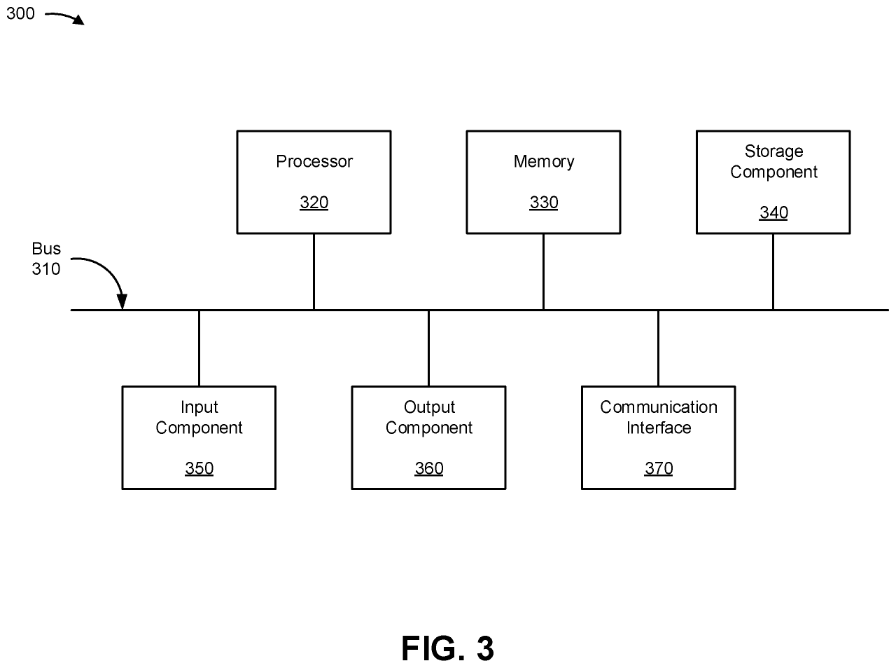

FIG. 3 is a diagram of example components of a device 300. Device 300 may correspond to implant customization platform 210, computing resource 215, user device 230, data storage device 240, medical imaging device 250, manufacturing device 260, and/or the like. In some implementations, implant customization platform 210, computing resource 215, user device 230, data storage device 240, medical imaging device 250, and/or manufacturing device 260 may include one or more devices 300 and/or one or more components of device 300. As shown in FIG. 3, device 300 may include a bus 310, a processor 320, a memory 330, a storage component 340, an input component 350, an output component 360, and a communication interface 370.

Bus 310 includes a component that permits communication among multiple components of device 300. Processor 320 is implemented in hardware, firmware, and/or a combination of hardware and software. Processor 320 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 320 includes one or more processors capable of being programmed to perform a function. Memory 330 includes a random access memory (RAM), a read only memory (ROM), and/or another type of dynamic or static storage device (e.g., a flash memory, a magnetic memory, and/or an optical memory) that stores information and/or instructions for use by processor 320.

Storage component 340 stores information and/or software related to the operation and use of device 300. For example, storage component 340 may include a hard disk (e.g., a magnetic disk, an optical disk, and/or a magneto-optic disk), a solid state drive (SSD), a compact disc (CD), a digital versatile disc (DVD), a floppy disk, a cartridge, a magnetic tape, and/or another type of non-transitory computer-readable medium, along with a corresponding drive.

Input component 350 includes a component that permits device 300 to receive information, such as via user input (e.g., a touch screen display, a keyboard, a keypad, a mouse, a button, a switch, and/or a microphone). Additionally, or alternatively, input component 350 may include a component for determining location (e.g., a global positioning system (GPS) component) and/or a sensor (e.g., an accelerometer, a gyroscope, an actuator, another type of positional or environmental sensor, and/or the like). Output component 360 includes a component that provides output information from device 300 (via, e.g., a display, a speaker, a haptic feedback component, an audio or visual indicator, and/or the like).

Communication interface 370 includes a transceiver-like component (e.g., a transceiver, a separate receiver, a separate transmitter, and/or the like) that enables device 300 to communicate with other devices, such as via a wired connection, a wireless connection, or a combination of wired and wireless connections. Communication interface 370 may permit device 300 to receive information from another device and/or provide information to another device. For example, communication interface 370 may include an Ethernet interface, an optical interface, a coaxial interface, an infrared interface, a radio frequency (RF) interface, a universal serial bus (USB) interface, a Wi-Fi interface, a cellular network interface, and/or the like.

Device 300 may perform one or more processes described herein. Device 300 may perform these processes based on processor 320 executing software instructions stored by a non-transitory computer-readable medium, such as memory 330 and/or storage component 340. As used herein, the term “computer-readable medium” refers to a non-transitory memory device. A memory device includes memory space within a single physical storage device or memory space spread across multiple physical storage devices.

Software instructions may be read into memory 330 and/or storage component 340 from another computer-readable medium or from another device via communication interface 370. When executed, software instructions stored in memory 330 and/or storage component 340 may cause processor 320 to perform one or more processes described herein. Additionally, or alternatively, hardware circuitry may be used in place of or in combination with software instructions to perform one or more processes described herein. Thus, implementations described herein are not limited to any specific combination of hardware circuitry and software.

The number and arrangement of components shown in FIG. 3 are provided as an example. In practice, device 300 may include additional components, fewer components, different components, or differently arranged components than those shown in FIG. 3. Additionally, or alternatively, a set of components (e.g., one or more components) of device 300 may perform one or more functions described as being performed by another set of components of device 300.

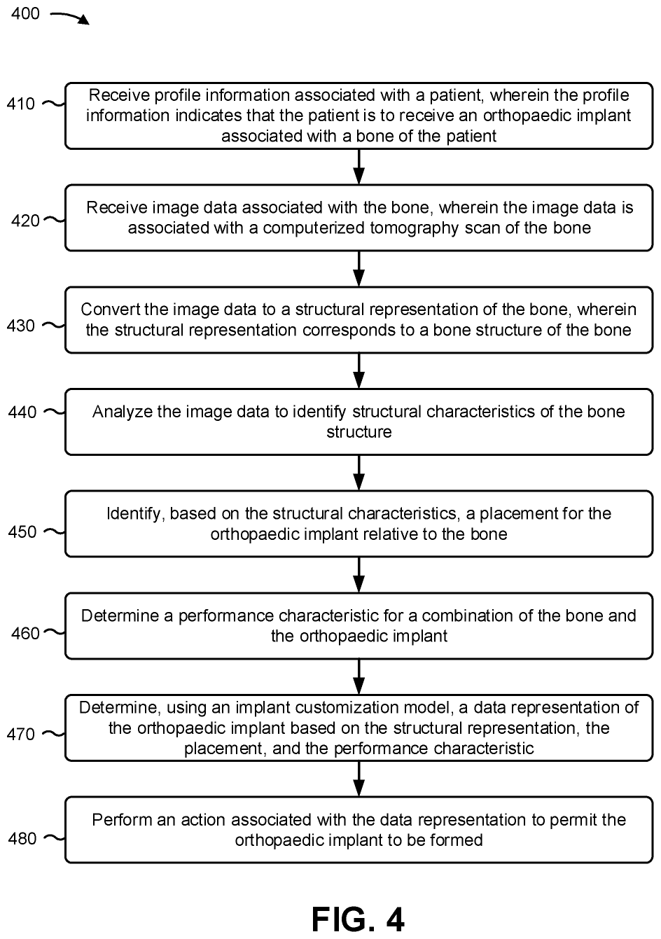

FIG. 4 is a flowchart of an example process 400 for customizing an orthopaedic implant. In some implementations, one or more process blocks of FIG. 4 may be performed by an implant customization platform (e.g., implant customization platform 210). In some implementations, one or more process blocks of FIG. 4 may be performed by another device or a group of devices separate from or including the implant customization platform, such as a user device (e.g., user device 230), a data storage device (e.g., data storage device 240), a medical imaging device (e.g., medical imaging device 250), a manufacturing device (e.g., manufacturing device 260), and/or the like.

As shown in FIG. 4, process 400 may include receiving profile information associated with a patient, wherein the profile information indicates that the patient is to receive an orthopaedic implant associated with a bone of the patient (block 410). For example, the implant customization platform (e.g., using processor 320, memory 330, storage component 340, input component 350, output component 360, communication interface 370 and/or the like) may receive profile information associated with a patient, as described above. In some implementations, the profile information indicates that the patient is to receive an orthopaedic implant associated with a bone of the patient.

As further shown in FIG. 4, process 400 may include receiving image data associated with the bone, wherein the image data is associated with a computed tomography scan of the bone (block 420). For example, the implant customization platform (e.g., using processor 320, memory 330, storage component 340, input component 350, output component 360, communication interface 370 and/or the like) may receive image data associated with the bone, as described above. In some implementations, the image data is associated with a computed tomography scan of the bone.

As further shown in FIG. 4, process 400 may include converting the image data to a structural representation of the bone, wherein the structural representation corresponds to a bone structure of the bone (block 430). For example, the implant customization platform (e.g., using processor 320, memory 330, storage component 340, input component 350, output component 360, communication interface 370 and/or the like) may convert the image data to a structural representation of the bone, as described above. In some implementations, the structural representation corresponds to a bone structure of the bone.

As further shown in FIG. 4, process 400 may include analyzing the image data to identify structural characteristics of the bone structure (block 440). For example, the implant customization platform (e.g., using processor 320, memory 330, storage component 340, input component 350, output component 360, communication interface 370 and/or the like) may analyze the image data to identify structural characteristics of the bone structure, as described above.

As further shown in FIG. 4, process 400 may include identifying, based on the structural characteristics, a placement for the orthopaedic implant relative to the bone (block 450). For example, the implant customization platform (e.g., using processor 320, memory 330, storage component 340, input component 350, output component 360, communication interface 370 and/or the like) may identify, based on the structural characteristics, a placement for the orthopaedic implant relative to the bone, as described above.

As further shown in FIG. 4, process 400 may include determining a performance characteristic for a combination of the bone and the orthopaedic implant (block 460). For example, the implant customization platform (e.g., using processor 320, memory 330, storage component 340, input component 350, output component 360, communication interface 370 and/or the like) may determine a performance characteristic for a combination of the bone and the orthopaedic implant, as described above.

As further shown in FIG. 4, process 400 may include determining, using an implant customization model, a data representation of the orthopaedic implant based on the structural representation, the placement, and the performance characteristic (block 470). For example, the implant customization platform (e.g., using processor 320, memory 330, storage component 340, input component 350, output component 360, communication interface 370 and/or the like) may determine, using an implant customization model, a data representation of the orthopaedic implant based on the structural representation, the placement, and the performance characteristic, as described above.

As further shown in FIG. 4, process 400 may include performing an action associated with the data representation to permit the orthopaedic implant to be formed (block 480). For example, the implant customization platform (e.g., using processor 320, memory 330, storage component 340, input component 350, output component 360, communication interface 370 and/or the like) may perform an action associated with the data representation to permit the orthopaedic implant to be formed, as described above.

Process 400 may include additional implementations, such as any single implementation or any combination of implementations described below and/or in connection with one or more other processes described elsewhere herein.

In a first implementation, the implant customization platform, when converting the image data to the structural representation, may determine respective graphical values of voxels of the image data; convert the respective graphical values to corresponding property values for the structural representation; and generate the structural representation from the property values. In a second implementation, alone or in combination with the first implementation, the placement is identified based on a type of orthopaedic implant, wherein the type of orthopaedic implant is identified in the profile information associated with the patient.

In a third implementation, alone or in combination with one or more of the first and second implementations, the performance characteristic includes at least one of: an ability of the combination of the bone and the orthopaedic implant to withstand a threshold level of force, an ability of the combination of the bone and the orthopaedic implant to withstand a threshold fatigue cycling, an ability to prevent the orthopaedic implant from causing a threshold level of stress shielding of the bone, a porosity of the orthopaedic implant, or an ability to administer a substance from the orthopaedic implant into the patient.

In a fourth implementation, alone or in combination with one or more of the first through third implementations, the performance characteristic is based on a patient characteristic of the patient, wherein the patient characteristic includes at least one of: an age of the patient, a bone mineral density of the patient, a sex of the patient, a weight of the patient, an expected load on the combination of the bone and the orthopaedic implant, or an expected load on a skeletal structure of the patient.

In a fifth implementation, alone or in combination with one or more of the first through fourth implementations, the bone is associated with a joint of the patient, wherein the orthopaedic implant is configured to provide structural support for the joint. In a sixth implementation, alone or in combination with one or more of the first through fifth implementations, the bone is associated with a spine of the patient, wherein the orthopaedic implant is configured to be received between vertebrae of the spine to provide structural support to the spine.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

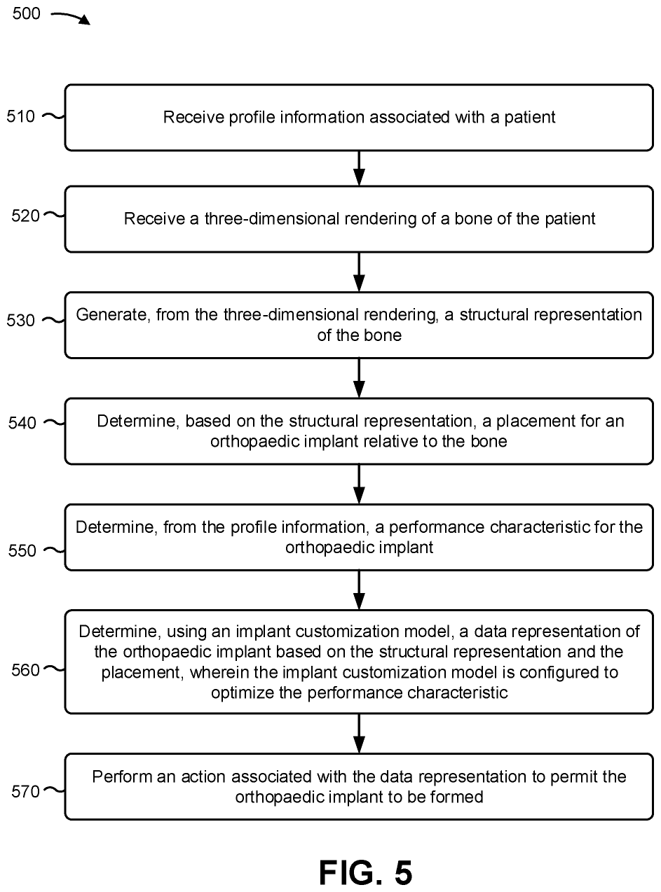

FIG. 5 is a flowchart of an example process 500 for customizing an orthopaedic implant. In some implementations, one or more process blocks of FIG. 5 may be performed by an implant customization platform (e.g., implant customization platform 210). In some implementations, one or more process blocks of FIG. 5 may be performed by another device or a group of devices separate from or including the implant customization platform, such as a user device (e.g., user device 230), a data storage device (e.g., data storage device 240), a medical imaging device (e.g., medical imaging device 250), a manufacturing device (e.g., manufacturing device 260), and/or the like.

As shown in FIG. 5, process 500 may include receiving profile information associated with a patient (block 510). For example, the implant customization platform (e.g., using processor 320, memory 330, storage component 340, input component 350, output component 360, communication interface 370 and/or the like) may receive profile information associated with a patient, as described above.

As further shown in FIG. 5, process 500 may include receiving a three-dimensional rendering of a bone of the patient (block 520). For example, the implant customization platform (e.g., using processor 320, memory 330, storage component 340, input component 350, output component 360, communication interface 370 and/or the like) may receive a three-dimensional rendering of a bone of the patient, as described above.

As further shown in FIG. 5, process 500 may include generating, from the three-dimensional rendering, a structural representation of the bone (block 530). For example, the implant customization platform (e.g., using processor 320, memory 330, storage component 340, input component 350, output component 360, communication interface 370 and/or the like) may generate, from the three-dimensional rendering, a structural representation of the bone, as described above.

As further shown in FIG. 5, process 500 may include determining, based on the structural representation, a placement for an orthopaedic implant relative to the bone (block 540). For example, the implant customization platform (e.g., using processor 320, memory 330, storage component 340, input component 350, output component 360, communication interface 370 and/or the like) may determine, based on the structural representation, a placement for an orthopaedic implant relative to the bone, as described above.

As further shown in FIG. 5, process 500 may include determining, from the profile information, a performance characteristic for the orthopaedic implant (block 550). For example, the implant customization platform (e.g., using processor 320, memory 330, storage component 340, input component 350, output component 360, communication interface 370 and/or the like) may determine, from the profile information, a performance characteristic for the orthopaedic implant, as described above.

As further shown in FIG. 5, process 500 may include determining, using an implant customization model, a data representation of the orthopaedic implant based on the structural representation and the placement, wherein the implant customization model is configured to optimize the performance characteristic (block 560). For example, the implant customization platform (e.g., using processor 320, memory 330, storage component 340, input component 350, output component 360, communication interface 370 and/or the like) may determine, using an implant customization model, a data representation of the orthopaedic implant based on the structural representation and the placement, as described above. In some implementations, the implant customization model is configured to optimize the performance characteristic.

As further shown in FIG. 5, process 500 may include performing an action associated with the data representation to permit the orthopaedic implant to be formed (block 570). For example, the implant customization platform (e.g., using processor 320, memory 330, storage component 340, input component 350, output component 360, communication interface 370 and/or the like) may perform an action associated with the data representation to permit the orthopaedic implant to be formed, as described above.

Process 500 may include additional implementations, such as any single implementation or any combination of implementations described below and/or in connection with one or more other processes described elsewhere herein. In a first implementation, the three-dimensional rendering is generated from images associated with a computed tomography scan of the bone.

In a second implementation, alone or in combination with the first implementation, the implant customization platform, when generating the structural representation, may determine respective graphical values of voxels of the three-dimensional rendering, convert the respective graphical values to corresponding property values for the structural representation, and generate the structural representation from the property values, wherein the structural representation is indicative of a bone structure of the bone.

In a third implementation, alone or in combination with one or more of the first and second implementations, the performance characteristic includes at least one of: an ability of the combination of the bone and the orthopaedic implant to withstand a threshold level of force, an ability of the combination of the bone and the orthopaedic implant to withstand a threshold fatigue cycling, an ability to prevent the orthopaedic implant from causing a threshold level of stress shielding of the bone, a porosity of the orthopaedic implant, or an ability to administer a substance from the orthopaedic implant into the patient.

In a fourth implementation, alone or in combination with one or more of the first through third implementations, the profile information includes at least one of: an age of the patient, a bone mineral density of the patient, a sex of the patient, a weight of the patient, an expected load on the combination of the bone and the orthopaedic implant, or an expected load on a skeletal structure of the patient.

In a fifth implementation, alone or in combination with one or more of the first through fourth implementations, the orthopaedic implant is at least one of: an arthroplasty implant or an interbody spine implant.

In a sixth implementation, alone or in combination with one or more of the first through fifth implementations, the implant customization platform, when performing the action, may store the data representation in a data structure of a manufacturing device, wherein the data representation can be used by the manufacturing device to form the orthopaedic implant, provide the data representation to the manufacturing device to cause the manufacturing device to form the orthopaedic implant, or provide the data representation to a user device, wherein the data representation can be used to present a graphical representation of the orthopaedic implant via a display of the user device.

Although FIG. 5 shows example blocks of process 500, in some implementations, process 500 may include additional blocks, fewer blocks, different blocks, or differently arranged blocks than those depicted in FIG. 5. Additionally, or alternatively, two or more of the blocks of process 500 may be performed in parallel.

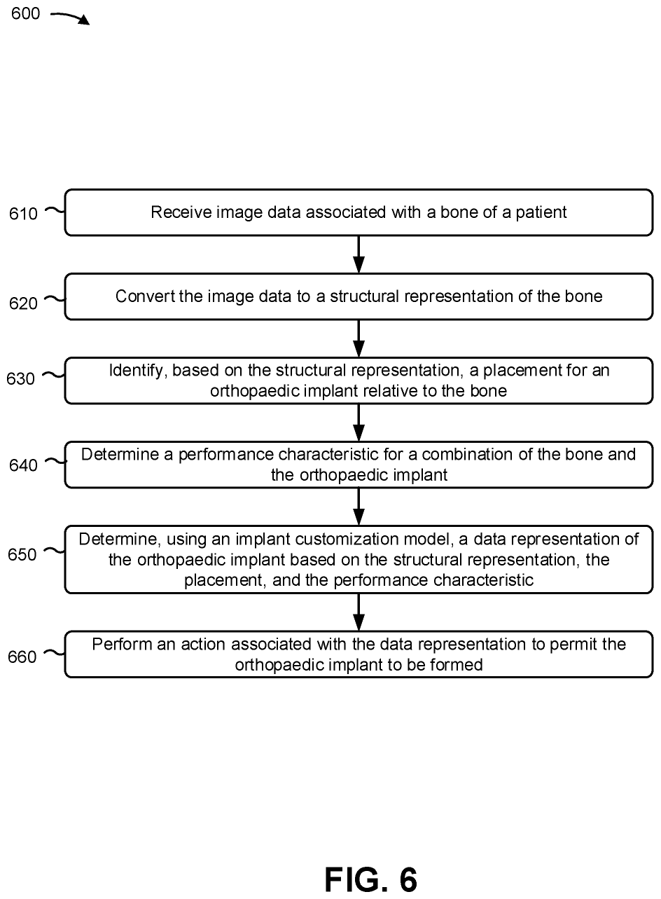

FIG. 6 is a flowchart of an example process 600 for customizing an orthopaedic implant. In some implementations, one or more process blocks of FIG. 6 may be performed by an implant customization platform (e.g., implant customization platform 210). In some implementations, one or more process blocks of FIG. 6 may be performed by another device or a group of devices separate from or including the implant customization platform, such as a user device (e.g., user device 230), a data storage device (e.g., data storage device 240), a medical imaging device (e.g., medical imaging device 250), a manufacturing device (e.g., manufacturing device 260), and/or the like.

As shown in FIG. 6, process 600 may include receiving image data associated with a bone of a patient (block 610). For example, the implant customization platform (e.g., using processor 320, memory 330, storage component 340, input component 350, output component 360, communication interface 370 and/or the like) may receive image data associated with a bone of a patient, as described above.

As further shown in FIG. 6, process 600 may include converting the image data to a structural representation of the bone (block 620). For example, the implant customization platform (e.g., using processor 320, memory 330, storage component 340, input component 350, output component 360, communication interface 370 and/or the like) may convert the image data to a structural representation of the bone, as described above.

As further shown in FIG. 6, process 600 may include identifying, based on the structural representation, a placement for an orthopaedic implant relative to the bone (block 630). For example, the implant customization platform (e.g., using processor 320, memory 330, storage component 340, input component 350, output component 360, communication interface 370 and/or the like) may identify, based on the structural representation, a placement for an orthopaedic implant relative to the bone, as described above.

As further shown in FIG. 6, process 600 may include determining a performance characteristic for a combination of the bone and the orthopaedic implant (block 640). For example, the implant customization platform (e.g., using processor 320, memory 330, storage component 340, input component 350, output component 360, communication interface 370 and/or the like) may determine a performance characteristic for a combination of the bone and the orthopaedic implant, as described above.

As further shown in FIG. 6, process 600 may include determining, using an implant customization model, a data representation of the orthopaedic implant based on the structural representation, the placement, and the performance characteristic (block 650). For example, the implant customization platform (e.g., using processor 320, memory 330, storage component 340, input component 350, output component 360, communication interface 370 and/or the like) may determine, using an implant customization model, a data representation of the orthopaedic implant based on the structural representation, the placement, and the performance characteristic, as described above.

As further shown in FIG. 6, process 600 may include performing an action associated with the data representation to permit the orthopaedic implant to be formed (block 660). For example, the implant customization platform (e.g., using processor 320, memory 330, storage component 340, input component 350, output component 360, communication interface 370 and/or the like) may perform an action associated with the data representation to permit the orthopaedic implant to be formed, as described above.

Process 600 may include additional implementations, such as any single implementation or any combination of implementations described below and/or in connection with one or more other processes described elsewhere herein.

In a first implementation, the image data is associated with a three-dimensional rendering of the bone, wherein the three-dimensional rendering is generated from images associated with a computed tomography scan of the bone.

In a second implementation, alone or in combination with the first implementation, the implant customization platform, when converting the image data to the structural representation, may determine respective graphical values of voxels of the image data, convert the respective graphical values to corresponding property values for the structural representation, and generate the structural representation from the property values, wherein the structural representation is indicative of a structure of the bone.

In a third implementation, alone or in combination with one or more of the first and second implementations, the performance characteristic includes at least one of: an ability of the combination of the bone and the orthopaedic implant to withstand a threshold level of force, an ability of the combination of the bone and the orthopaedic implant to withstand a threshold fatigue cycling, an ability to prevent the orthopaedic implant from causing a threshold level of stress shielding of the bone, a porosity of the orthopaedic implant, or an ability to administer a substance from the orthopaedic implant into the patient. In a fourth implementation, alone or in combination with one or more of the first through third implementations, the implant customization model is configured to optimize the performance characteristic according to a topology optimization model.

In a fifth implementation, alone or in combination with one or more of the first through fourth implementations, the implant customization platform, when performing the action, may store the data representation in a data structure, wherein the data representation can be used by the manufacturing device to form the orthopaedic implant; provide the data representation to the manufacturing device to cause the manufacturing device to form the orthopaedic implant; or provide the data representation to a user device, wherein the data representation can be used to present a graphical representation of the orthopaedic implant via a display of the user device.

Although FIG. 6 shows example blocks of process 600, in some implementations, process 600 may include additional blocks, fewer blocks, different blocks, or differently arranged blocks than those depicted in FIG. 6. Additionally, or alternatively, two or more of the blocks of process 600 may be performed in parallel.

The foregoing disclosure provides illustration and description but is not intended to be exhaustive or to limit the implementations to the precise forms disclosed. Modifications and variations may be made in light of the above disclosure or may be acquired from practice of the implementations.

As used herein, the term “component” is intended to be broadly construed as hardware, firmware, and/or a combination of hardware and software.

As used herein, satisfying a threshold may, depending on the context, refer to a value being greater than the threshold, more than the threshold, higher than the threshold, greater than or equal to the threshold, less than the threshold, fewer than the threshold, lower than the threshold, less than or equal to the threshold, equal to the threshold, or the like.

Certain user interfaces have been described herein and/or shown in the figures. A user interface may include a graphical user interface, a non-graphical user interface, a text-based user interface, and/or the like. A user interface may provide information for display. In some implementations, a user may interact with the information, such as by providing input via an input component of a device that provides the user interface for display. In some implementations, a user interface may be configurable by a device and/or a user (e.g., a user may change the size of the user interface, information provided via the user interface, a position of information provided via the user interface, etc.). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

It will be apparent that systems and/or methods described herein may be implemented in different forms of hardware, firmware, or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be designed to implement the systems and/or methods based on the description herein.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, etc.), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A method, comprising:
receiving, by a device, profile information associated with a patient,
wherein the profile information indicates that the patient is to receive an orthopaedic implant associated with a bone of the patient;
receiving, by the device, image data associated with the bone,
wherein the image data is associated with a computed tomography scan of the bone;
converting, by the device, the image data to a structural representation of the bone,
wherein the structural representation corresponds to a bone structure of the bone;
analyzing, by the device, the image data to identify structural characteristics of the bone structure;
identifying, by the device and based on the structural characteristics, a placement for the orthopaedic implant relative to the bone;
determining, by the device, a performance characteristic for a combination of the bone and the orthopaedic implant;
determining, by the device and using an implant customization model, a data representation of the orthopaedic implant based on the structural representation, the placement, and the performance characteristic; and
performing, by the device, an action associated with the data representation to permit the orthopaedic implant to be formed.

2. The method of claim 1, wherein converting the image data to the structural representation comprises:
determining respective graphical values of voxels of the image data;
converting the respective graphical values to corresponding property values for the structural representation; and
generating the structural representation from the property values.

3. The method of claim 1, wherein the placement is identified based on a type of orthopaedic implant,
wherein the type of orthopaedic implant is identified in the profile information associated with the patient.

4. The method of claim 1, wherein the performance characteristic includes at least one of:
an ability of the combination of the bone and the orthopaedic implant to withstand a threshold level of force,
an ability of the combination of the bone and the orthopaedic implant to withstand a threshold fatigue cycling,
an ability to prevent the orthopaedic implant from causing a threshold level of stress shielding of the bone,
a porosity of the orthopaedic implant, or
an ability to administer a substance from the orthopaedic implant into the patient.

5. The method of claim 1, wherein the performance characteristic is based on a patient characteristic of the patient,
wherein the patient characteristic includes at least one of,
an age of the patient,
a bone mineral density of the patient,
a sex of the patient,
a weight of the patient,
an expected load on the combination of the bone and the orthopaedic implant, or
an expected load on a skeletal structure of the patient.

6. The method of claim 1, wherein the bone is associated with a joint of the patient,
wherein the orthopaedic implant is configured to provide structural support for the joint.

7. The method of claim 1, wherein the bone is associated with a spine of the patient,
wherein the orthopaedic implant is configured to be received between vertebrae of the spine to provide structural support to the spine.

8. A device, comprising:
one or more memories; and
one or more processors communicatively coupled to the one or more memories, to:
receive profile information associated with a patient;
receive a three-dimensional rendering of a bone of the patient;
generate, from the three-dimensional rendering, a structural representation of the bone;
determine, based on the structural representation, a placement for an orthopaedic implant relative to the bone;
determine, from the profile information, a performance characteristic for the orthopaedic implant;
determine, using an implant customization model, a data representation of the orthopaedic implant based on the structural representation and the placement,
wherein the implant customization model is configured to optimize the performance characteristic; and
perform an action associated with the data representation to permit the orthopaedic implant to be formed.

9. The device of claim 8, wherein the three-dimensional rendering is generated from images associated with a computed tomography scan of the bone.

10. The device of claim 8, wherein the one or more processors, when generating the structural representation, are to:
determine respective graphical values of voxels of the three-dimensional rendering;
convert the respective graphical values to corresponding property values for the structural representation; and
generate the structural representation from the property values,
wherein the structural representation is indicative of a bone structure of the bone.

11. The device of claim 8, wherein the performance characteristic includes at least one of:
an ability of the combination of the bone and the orthopaedic implant to withstand a threshold level of force,
an ability of the combination of the bone and the orthopaedic implant to withstand a threshold fatigue cycling,
an ability to prevent the orthopaedic implant from causing a threshold level of stress shielding of the bone,
a porosity of the orthopaedic implant, or
an ability to administer a substance from the orthopaedic implant into the patient.

12. The device of claim 8, wherein the profile information includes at least one of:
an age of the patient,
a bone mineral density of the patient,
a sex of the patient,
a weight of the patient,
an expected load on the combination of the bone and the orthopaedic implant, or
an expected load on a skeletal structure of the patient.

13. The device of claim 8, wherein the orthopaedic implant is at least one of:
an arthroplasty implant, or
an interbody spine implant.

14. The device of claim 8, wherein the one or more processors, when performing the action, are to at least one of:
store the data representation in a data structure of a manufacturing device,
wherein the data representation can be used by the manufacturing device to form the orthopaedic implant,
provide the data representation to the manufacturing device to cause the manufacturing device to form the orthopaedic implant,
store the data representation for use in generating an orthopaedic implant for a group of patients associated with the patient, or
provide the data representation to a user device,
wherein the data representation can be used to present a graphical representation of the orthopaedic implant via a display of the user device.

15. A non-transitory computer-readable medium storing instructions, the instructions comprising:
one or more instructions that, when executed by one or more processors, cause the one or more processors to:
receive image data associated with a bone of a patient;
convert the image data to a structural representation of the bone;
identify, based on the structural representation, a placement for an orthopaedic implant relative to the bone;
determine a performance characteristic for a combination of the bone and the orthopaedic implant;
determine, using an implant customization model, a data representation of the orthopaedic implant based on the structural representation, the placement, and the performance characteristic; and
perform an action associated with the data representation to permit the orthopaedic implant to be formed.

16. The non-transitory computer-readable medium of claim 15, wherein the image data is associated with a three-dimensional rendering of the bone,
wherein the three-dimensional rendering is generated from images associated with a computed tomography scan of the bone.

17. The non-transitory computer-readable medium of claim 15, wherein the one or more instructions, that cause the one or more processors to convert the image data to the structural representation, cause the one or more processors to:
determine respective graphical values of voxels of the image data;
convert the respective graphical values to corresponding property values for the structural representation; and
generate the structural representation from the property values,
wherein the structural representation is indicative of a structure of the bone.

18. The non-transitory computer-readable medium of claim 15, wherein the performance characteristic includes at least one of:
an ability of the combination of the bone and the orthopaedic implant to withstand a threshold level of force,
an ability of the combination of the bone and the orthopaedic implant to withstand a threshold fatigue cycling,
an ability to prevent the orthopaedic implant from causing a threshold level of stress shielding of the bone,
a porosity of the orthopaedic implant, or
an ability to administer a substance from the orthopaedic implant into the patient.

19. The non-transitory computer-readable medium of claim 15, wherein the implant customization model is configured to optimize the performance characteristic according to a topology optimization model.

20. The non-transitory computer-readable medium of claim 15, wherein the one or more instructions, that cause the one or more processors to perform the action, cause the one or more processors to at least one of:
store the data representation in a data structure,
wherein the data representation can be used by a manufacturing device to form the orthopaedic implant,
provide the data representation to the manufacturing device to cause the manufacturing device to form the orthopaedic implant, or
provide the data representation to a user device,
wherein the data representation can be used to present a graphical representation of the orthopaedic implant via a display of the user device.
